(19) United States
(12) Patent Application Publication
(10) Pub. No.: US 2010/0316700 A1
(43) Pub. Date: Dec. 16, 2010

(54) SYSTEMIC ADMINISTRATION OF LIPOSOMAL REDUCED GLUTATHIONE FOR THE DECORPORATION OF RADIOACTIVE MATERIALS AND NON-RADIOACTIVE MERCURY

(76) Inventor: F. Timothy Guilford, Palo Alto, CA (US)

Correspondence Address: BROOKE SCHUMM III;Daneker, McIntire, Schumm, Prince, Goldstein et al, ONE NORTH CHARLES STREET, SUITE 2450, BALTIMORE, MD, 21201, US

(21) Appl. No.: 12/702,283

(22) Filed: Feb. 8, 2010

Related U.S. Application Data
(63) Continuation-in-part of application No. 10/289,934, filed on Nov. 7, 2002.
Application No. 10/289,934 is a continuation-in-part of application No. 11/230,277, filed on Sep. 20, 2005.
(60) Provisional application No. 61/150,726, filed on Feb. 6, 2009.

(51) Int. Cl. A61K 38/06; A61K 9/127; A61K 33/04; A61K 38/30; A61P 39/04; A61P 39/00

(52) U.S. Cl. 424/450; 514/21.9; 424/702; 514/8.6

(57) ABSTRACT

The invention is for the combination and related methods of liposomal reduced glutathione in oral, inhaled, or intravenous, or glutathione inhaled or intravenous, to ameliorate the effects of exposure to or ingestion of radiation or radioactive materials, as treatment to ameliorate the toxic effects of radiation exposure or ingestion and to facilitate the decorporation of radioactive materials such as Co-60. The invention may also be used in conjunction with a decorporation agent such as DTPA, EDTA, DMPS or penicillamine to ameliorate the toxic effects of exposure or ingestion of radioactive agents or other toxic metals.

### CONTINUATION DATA

For US and regional purposes, when filed as a utility application for a U.S. utility patent or the regional or foreign equivalent in each respective region or nation, this application is a continuation in part of U.S. utility application Ser. No. 10/289,934 filed on Nov. 7, 2002 published as 20040022873 entitled “Systemic administration of NAC as an adjunct in the treatment of bioterror exposures such as anthrax, small pox or radiation and for vaccination prophylaxis, and use in combination with DHEA for the treatment of smallpox and other viruses,” and U.S. utility application Ser. No. 11/230,277 filed on Sep. 20, 2005 entitled “Combination And Method Using EDTA Combined With Glutathione In The Reduced State Encapsulated In A Liposome To Facilitate The Method Of Delivery Of The Combination As An Oral, Topical, Intraoral Or Transmucosal, For Anti-Thrombin Effect And For Anti-Platelet Aggregation And Measurement Of Efficacy,” U.S. Provisional Application 61/150,726 and of a provisional application being filed this day with the title of this invention, all of which are incorporated by reference herein.

### SUMMARY

The invention is for liposomal reduced glutathione which can be administered in oral, topical, inhaled, or intravenous form, or combinations for the decorporation of dirty bomb materials, including Cobalt (Co-60), Strontium (Sr)-90, yttrium (Y)-90, cesium (Cs)-137, iridium (Ir)-192, americium (Am)-241, iodine (I)-125 and 131, uranium (U)-233, 234, 235, and 238, plutonium (Pu)-239, radium (Ra)-226, tritium (hydrogen-3 or H-3), phosphorus (P)-32 and palladium (Pd)-103, and a non-radioactive toxic metal in the form of methyl mercury. Collectively, that list of radioactive materials used in radioactive dispersal devices (“RDD”), without the Co-60, will be referred to as “RDD Radiotoxic Materials” and in the claims as “RDD radiotoxic materials.” and a non-radioactive toxic metal in the form of mercury or methyl mercury. The invention may also be used in conjunction with a decorporation agent such as DMPS, DTPA, EDTA, or penicillamine to ameliorate the toxic effects of exposure or ingestion of radioactive agents or other toxic metals. Recent, novel information has demonstrated the surprising effect that liposomal reduced glutathione is capable of increasing the decorporation of Cobalt-60 (60Co), which has been identified as a possible radiation source known as a “dirty bomb”. A recent publication also shows the surprising effect of approximately 100-fold

### TECHNICAL FIELD

The present invention relates to the treatment and removal of materials from sources that emit alpha, beta and gamma radiation using liposomal reduced glutathione oral, inhaled, transmucosal, intravenous or topical, by itself. Further, the invention proposes the combination of liposomal reduced glutathione capable of administration in a form oral, inhaled, transmucosal, intravenous or topical, in combination with a decorporation agent such as DMPS, DTPA, EDTA, or penicillamine. As part of this invention, a liposome encapsulation of reduced glutathione is used as both a systemic antioxidant and an agent for the decorporation of radionuclides.

The therapy is for lessening the cell and tissue damaging effects of radiation induced depletion of antioxidant defense alone or with a decorporation agent such as DTPA or with other metal mobilizing agents such as DMPS or DMSA or penicillamine.

In order to facilitate the removal of radionuclides from a mammal, liposomal reduced glutathione is administered for:
1) Decorporation of radioactive substances
2) lessening the tissue damaging effects of radiation
3) Enhancing the efficacy of other agents to lessen the tissue damaging effects of radiation.

The invention, liposomal reduced glutathione, may also be administered in combination with Insulin Growth Factor (IGF)-1 or DHEA as a combination to support the endocrine system after exposure to ionizing radiation.

### BACKGROUND OF THE INVENTION

Radioactivity is a characteristic of a material and radiation is the result of a radioactive material, often a naturally occurring or artificially generated material, or sometimes may be artificially generated radiation such as from an X-ray tube. The result of radioactivity is the generation particle emissions, including neutron, x-ray, alpha, beta or gamma particles. Alpha particles are considered high linear energy transfer (LET) particles and deliver substantive damage to DNA in the form of double stranded DNA breaks, which are very difficult for cells to repair properly. Gamma rays, and x rays, in contrast are low LET particles and operate by the generation of radiolysis of water generating hydroxyl free radicals in the vicinity of DNA causing single strand and double stranded breaks following a linear-quadratic curve of cell survival v. dose, culminating in a loss of reproductive integrity of the cancer cells Likewise beta particles, though low in linear energy transfer can cause double stranded breaks and destroy DNA through clusters of single stranded breaks which can be made permanent by oxygen fixation in non-hypoxic environments.

The radiation produced by radioactive materials provides a low-cost way to disinfect food, sterilize medical equipment, treat certain kinds of cancer, find oil, build sensitive smoke detectors, and provide other critical services in our economy. As a result, significant amounts of radioactive materials are stored in laboratories, food irradiation plants, oil drilling facilities, medical centers, and many other sites. As such, radioactive materials are available for both their beneficial uses, and for non-beneficial uses such as terrorism or nuclear warfare, although the latter may be less dependent on fission or fusion reaction. Since Sep. 11, 2001, there has been an increasing concern that terrorist events would recur and could escalate to the use of radioactive materials. Delivery of a general radiation releasing event such as an atomic bomb requires a set of complex delivery systems that decrease the probability that this type of device would be delivered by a terrorist. However, concern over the creation of what is termed a “dirty bomb” has received an increasingly high priority for response. As described by the Center for Disease Control website(1), a dirty bomb is a mix of explosives, such as dynamite, with radioactive powder or pellets. When the dynamite or other explosives are set off, the blast carries radioactive material into the surrounding area. A “dirty bomb” is created by combining radioactive material and a conventional explosive such as dynamite to aerosolize the material in the local environment. This type of device has also been called a radiological dispersal device (RDD). A number of materials are considered as candidates for use in a RDD based on either there availability from industrial sources or other characteristics. As an example, the world-wide use of the radiation releasing material Cobalt-60 (Co-60 or 60Co) in conventional and medical applications is often cited as an example of a material that could be used to form a dirty bomb. Strontium (Sr)-90, yttrium (Y)-90, cesium (Cs)-137, iridium (Ir)-192, americium (Am)-241, iodine (I)-125 and 131, uranium (U)-233, 234, 235, and 238, plutonium (Pu)-239, radium (Ra)-226, tritium (hydrogen-3 or H-3), phosphorus (P)-32 and palladium (Pd)-103 would generate the same kind of dirty bomb effect.

Breathing or swallowing the aerosolized dust from an RDD explosion can result in the inhalation and ingestion of radioactive particles. As the amount of material ingested or inhaled is likely to be less than that expected to cause immediate death, the damage from such exposures is likely to be due to the effects of prolonged exposure to the radiation releasing material in close proximity to the cells of the body, while inside the body. In contrast to an external exposure, which is brief, ingestion, inhalation or systemic intake of radioactive material results in a prolonged exposure, increasing the likelihood of damage to the body. The more quickly a radioactive material such as Co-60 or an RDD Radiotoxic Material is removed from the body, the fewer and less serious the health effects will be. A corollary to this observation is the concept that the longer the radioactive material stays in the body, the more difficult it becomes to remove the material (1). Further, the longer the agent is in the body, the more prolonged its side effects, including side effects related to systemic stress as opposed to the radioactivity per se, e.g., while initially, the damage is likely directly from ionization resulting from radioactive decay, as time passes, relatively more damage will result from combinations of impaired system functions or immune functions which functional impairments were caused by initial ionization damage. Materials and methods that facilitate the removal of radiation producing materials from the body fall in the class of “decorporation.” The broader class of nuclear, biological and chemical weapons are referred to as nuclear, biological and chemical (“NBC”) weapons.

### Background of Reduced Glutathione as Antioxidant and Detoxification Agent

Mammalian systems use the glutathione related antioxidant and detoxification system for removal of toxic materials. Glutathione helps support the antioxidant system and immune function during cell repair.

Because the present invention, liposomal reduced glutathione, provides a novel and unique method of delivering reduced glutathione to an individual in a form that can readily achieve systemic circulation, it is proposed as an invention to ameliorate both the acute effects of radiation such as radiation burns (sometimes referred to as deterministic effects) and delayed effects (sometimes referred to as random or stochastic effects) such as the delayed appearance of atherosclerosis in individuals exposed to ionizing radiation. The non-toxic nature of liposomal reduced glutathione makes it an especially attractive candidate for unsupervised treatment of the general population in case of radiological/nuclear emergency and for its continued administration after exposure to ionizing radiation. Low systemic availability due to poor absorption after oral ingestion of non-liposomally formulated reduced glutathione limits its effective administration to the intravenous infusion route made this treatment option unavailable until now. We have developed a novel, stable formulation of reduced glutathione for oral delivery (sold as ReadiSorb™ liposomal reduced glutathione by YES, LLC of Palo Alto, Calif.) and report that liposomal encapsulation markedly enhances the cellular bioavailability of reduced glutathione, meaning the novel, stable formulation is both storable for long periods and survives oral administration, and as a result, intracellular glutathione is enhanced and achieves results not achievable by non-intravenous administration of either plain glutathione (in any form) or non-liposomal reduced glutathione (which is normally largely oxidized in the gastrointestinal tract). (See human absorption studies below).

Selenium bioavailability is associated with the availability and function of an enzyme that works with glutathione, glutathione peroxidase.(2, 3). As set forth in Chung, L. W. K. et al, “Prostate Cancer: Biology, Genetics, and the New Therapeutics,” chapter 21: Brooks, J. D. et al, “Chemoprevention of Prostate Cancer,” at p. 370 (Humana Press 2001), Selenium is an important essential trace element to the chemical function of glutathione peroxidase.
“We have recently analyzed serum selenium values in a nested case-control study of men enrolled in the Baltimore Longitudinal Study of Aging. Our findings replicate those of the HPFS in that men with very low serum selenium levels manifested the highest risk for prostate cancer (6). Intriguingly, in both studies, there appeared to be a threshold effect—that once a certain level of selenium was attained, no further reduction in risk occurred.
Selenium, an essential trace element, is incorporated into a small number of proteins in the form of selenocysteine. Selenoproteins include glutathione peroxidase, an enzyme which plays a critical role in defending against oxidative damage and is particularly effective at reducing lipid peroxides. In the past, GSTs have been suspected to function as “selenium-independent” glutathione peroxidases. It is tempting to speculate that selenium may attenuate prostatic carcinogenesis by compensating for the somatic inactivation of GSTP1 through increasing total glutathione peroxidase activity in prostatic cells.” “Prostate Cancer, supra, at p. 370

The inventors' recommend administration of Selenium 100 mcg to 200 mcg in addition to liposomal reduced glutathione to maintain and restore Selenium levels. Selenium may administered as organic forms of selenium, such as selenomethionine and selenocysteine, as well as the inorganic forms of the mineral, like sodium selenite and selenate. There is no need to over-administer selenium, but in its absence, the function of glutathione peroxidase and the liposomal reduced glutathione will be attenuated.

Glutathione has two forms, reduced glutathione as shown on the left of the chemical drawing below and oxidized glutathione shown on the right.

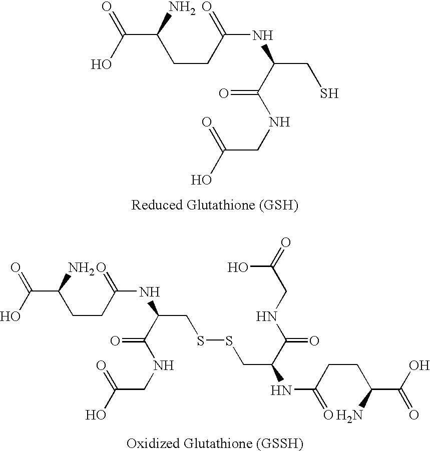

The reduced form is biologically active and ready for use. The oxidized form has already donated its electrons, so it is not effective as an antioxidant because the sulfur group which allows it to bind to metals is already bound to another oxidized glutathione molecule and is not available for use for detoxification. In plain glutathione, the reduced and oxidized forms stay in relative equilibrium. “Normal” reduced glutathione steadily oxidizes to an oxidized form, unless somehow protected, and does not usually survive the insult of the gastrointestinal tract. The present invention, liposomal reduced glutathione, provides a novel and unique method of delivering reduced glutathione to an individual in a form that can readily achieve systemic circulation; therefore the invention proposes a novel and easily administrable agent and method to facilitate decorporation individually and to be used in combination with additional agents of decorporation.

Internal contamination with radionuclides such as Co-60, or RDD Radiotoxic Materials can result in an acute illness from the radiation exposure, or, in a prolonged chronic illness as a result of exposure to relatively low levels of radiation. The prolonged exposure carries risks ranging from infection due to immune dysfunction in the days to weeks after an exposure to an increased risk of cancer to a longer-term effect such as atherosclerosis in later years. (4) The treatment after an internal contamination will be needed to help prevent the occurrence of acute health effects attributable to radiation exposure (termed ‘deterministic’ effects) and to restrict the likelihood of late health effects (termed ‘stochastic’ or random effects) such as cancers and some hereditable diseases.(5) While measures to prevent damage from radiation are important, diminishing the exposure time by facilitating the removal of the radionuclide from the body provides significant benefit in reducing the potential of illness after exposure. The availability for rapid administration of a radiation countermeasure to help reduce internal contamination would improve health outcomes for exposed populations as well as mitigate panic after the event. Early treatment is recommended and because of the ease of use in cases where a significant intake is suspected but may take time to confirm.(6) Availability of a non-toxic decorporation agent that could be administered as soon as possible after a radiological event, is critically important. Moreover, it is important that if too much of such a decorporation agent is consumed, there should not be toxicity from overdose of a decorporation or anti-radiation agent.

To date, the FDA approved agents for decorporation treatments have significant limitations. These agents include diethylenetriaminepentaacetic acid (DTPA)(7, 8), chelation therapy for transuranic radionuclides, potassium iodide (9) for radioisotopes of iodine, and hexacyanoferrate (II) called Prussian Blue(10) for radioisotopes of cesium and thallium. Few decorporation treatments have been suggested for other radionuclides.(11) No effective decorporation agents currently exist for Co-60, Ir-192, Po-210, Ra-226, and a number of other radioisotopes. It appears that different groups of radionuclides will require different synthetic receptors possessing specific functional groups. Natural receptors, which exist in diverse physicochemical forms, often possess versatile and superior chelation properties due to the presence of multiple functional groups, making them capable of binding dissimilar metal ions via multiple chelation mechanisms.

In biological systems, metal ions are strongly coordinated to aminothiol receptors including proteins and polypeptides. Tripeptide (γ-Glu-Cys-Gly) glutathione, one of the most ubiquitous small biological molecules found in almost every cell of the living organism at relatively high (millimolar) concentrations, exists in reduced and oxidized states (FIG. 1) and exhibits a multitude of biological functions. (12) Glutathione plays an important role in toxicology and homeostasis because of its ability to coordinate a variety of metal ions (13, 14), in part due to its cysteine thiol functionality. Among naturally occurring amino acids, the sulfur molecule of cysteine exhibits a unique ability to bind various metal ions, including Co(II)/Co(III), and Ir-192, Po-210, and Ra-226 and other RDD Radiotoxic Materials.(15) However, cysteine, using the large amino acid transport system in cell membranes, has been implicated as a carrier of metals, such as methylmercury, into cells.(16) The “free ride” means that the complex of glutathione and methylmercury can be eliminated by passive and active systems that do not interfere with the existing ion channels. Reduced glutathione is known to carry metals out of cells and out of the body through the liver system. Reduced glutathione is known to complex with metals such as mercury, forming metal-glutathione (GS—Hg—SG), which is similar to that of oxidized glutathione (GS—SG) and thereby gets a “free ride” across the liver cell membrane on the endogenous carrier for oxidized glutathione.(16) The versatile metal chelation properties and the low toxicity of reduced glutathione make it an attractive candidate agent for decorporation therapy. Because of its low systemic availability upon ingestion (17), reduced glutathione not formulated in conformance with this invention, requires intravenous (IV) administration, which prevents its unsupervised administration to the general public in the event of radiological/nuclear emergency. The reason for the low availability upon ingestion is that in the acidic environment of the stomach, the proton-donor/electron-acceptor of the stomach's acidic environment rapidly combines with reduced glutathione and limits its bioavailability. The inventor has developed a novel formulation of reduced glutathione (liposomal reduced glutathione, trade name ReadiSorb™ liposomal reduced glutathione sold by YES, LLC of Palo Alto, Calif.) (18) especially designed to maintain glutathione in the reduced state for a prolonged period and maximize gastrointestinal absorption of reduced glutathione.

Recent, novel information has demonstrated the surprising effect that liposomal reduced glutathione is capable of increasing the decorporation of Cobalt-60 (60Co), which has been identified as a possible radiation source known as a “dirty bomb”. See Case Example 1.

A surprising intracellular effect is reported by Dr. Gail Zeevalk of the College of New Jersey, “Liposomal glutathione for the replenishment and maintenance of intracellular glutathione in mesencephalic cultures, Society for Neuroscience, Poster, Session 246, Mechanisms of Neuroprotection II, (Chicago October 2009). The surprising effect confirmed the results in Rosenblat M, Volkova N, Coleman R, Aviram M. Anti-Oxidant And Anti-Atherogenic Properties Of Liposomal Glutathione: Studies In Vitro, And In The Atherosclerotic Apolipoprotein E-Deficient Mice, Atherosclerosis, 2007;195(2):e61-68.

In the recent article by Zeevalk, et al, “Liposomal glutathione for the replenishment and maintenance of intracellular glutathione in mesencephalic cultures, Society for Neuroscience, Poster, Session 246, Mechanisms of Neuroprotection II, (Chicago October 2009), Dr. Zeevalk ran a series of tests of this invention related to the unusual activity of the liposomal formulation of reduced glutathione invented by Dr. Guilford sold as ReadiSorb® liposomal reduced glutathione by Your Energy Systems, LLC. The study was directed at studying the ability of the liposomal reduced glutathione formulation to replenish intracellular levels of glutathione in mixed neuronal and glial mesencephalic cultures. After depletion of glutathione by 60% with a 30 minute pretreatment with diethyl maleate, repletion of intracellular glutathione was followed over 4 hours in a Krebs Ringer containing either no additives, reduced glutathione, amino acid substrates glutamine, cysteine and glycine (for optimal repletion) or various concentrations of non-liposomal glutathione or liposomal glutathione. In the absence of additives, no repletion of glutathione was observed. With liposomal glutathione, and non liposomal glutathione, intracellular glutathione was restored. However, liposomal glutathione in the formulation of this invention was found to be 100 times more potent than non-liposomal glutathione at restoring glutathione levels.

Surprisingly, one would not have expected in any way that Dr. Guilford's formulation to have an effect 100 times as potent intracellularly as non-liposomal glutathione. This is a surprising effect and illustrates the uniqueness of the liposomal reduced glutathione formulation and its contribution, not before achieved.

The ideal radio-protector or therapeutic agent must be safe for all populations at risk of radiation exposure, even with repeated doses (as needed), must be easily administered, must be rapidly effective, and must be chemically stable so it can be stored in available form for treatment.(19) The qualities of the present invention, liposomal reduced glutathione in a liquid for oral ingestion that has storage longevity corresponds with the described ideal characteristics for a radio-protector and decorporation agent.

Radioactive Elements of Concern

A number of materials, in addition to Co-60, have been identified as possible sources of dirty bomb material including Strontium (Sr)-90, yttrium (Y)-90, cesium (Cs)-137, iridium (Ir)-192, cobalt (Co)-60, americium (Am)-241, iodine (I)-125 and 131, uranium (U)-233, 234, 235, and 238, plutonium (Pu)-239, radium (Ra)-226, tritium (hydrogen-3 or H-3), phosphorus (P)-32 and palladium (Pd)-103. Collectively, that list of materials, without the Co-60, will be referred to as “RDD Radiotoxic Materials” and in the claims as “RDD radiotoxic materials.” Because some of these complex with body ligands in insoluble, and thus poorly excretable, forms in the body, some complex with decorporation agents in insoluble form and are thus poorly excretable, and others are excreted slowly because of other biochemical cycle, liposomal reduced glutathione speeds the decorporation so that the cell excretory process is more effective.

⁶⁰Co is cited as an example of the need for ways to ameliorate the effects of radiation exposure. ⁶⁰Co gamma radiation is used for sterilizing medical equipment and consumer products, radiation therapy for treating cancer patients, manufacturing plastics, and irradiating food. Radioactive materials are also widely used in university, corporate, and government research laboratories. As ⁶⁰Co is available at many sites, information about its removal is pertinent. It takes about 5.27 years for Co-60 to give off half of its radiation; this is called the half-life (20).

After exposure in mammals, ⁶⁰Co is excreted via urine and feces, but some of the material persists in the liver, kidneys and bone marrow, where the prolonged exposure to radiation has the potential to cause both acute and long term problems. The problems include decreased immune cell function leading to infection in the short term and cancer in the long term.

At present there is no method for the decorporation of ⁶⁰Co in humans as noted in a review of radionuclide exposure, which review, in an “Alphabetical List of Radioelement and Decorporation Treatment Summary” asserts that for treatment of internal contamination by cobalt, there is “nothing too good, but oral penicillamine worth trying.” (20A) Penicillamine could be tried, but it did not work in mice. Cobaltous DTPA reduced radioactive cobalt concentration by about ⅓ in mice, but it has never been tried in humans, requires intravenous infusion and it is not presently available (21).

DTPA, a polyaminopolycarboxylic acid similar to EDTA, calcium disodium ethylenediaminetetraacetate (CaNa(2)EDTA), calcium trisodium or zinc trisodium diethylenetriaminepentaacetate (CaNa(3)DTPA, ZnNa(3)DTPA) has been proposed as a chelator of ⁶⁰Co. Currently, DTPA is only available by injection and is not available in an oral (by mouth) form. It is possible, depending on the severity of the exposure that treatment with a chelator such as DTPA may be necessary on a daily basis for weeks or months after exposure.(1) An oral form of DTPA as provided in a liposomal encapsulation of DTPA would offer a significant advantage for the treatment for decorporation therapy.

The combination of liposomal reduced glutathione and the polyaminopolycarboxylic acid material EDTA is reviewed in Guilford, U.S. patent application Ser. No. 11/230,277 filed Sep. 20, 2005, and published in the U.S. as No. 20070065. The method of encapsulating DTPA in a liposome for oral consumption is included in this current patent application, by itself and in combination with liposomal reduced glutathione.

Cell studies have demonstrated that a decreased level of glutathione increases sensitivity to radiation (Meister A et al, “Intracellular cysteine and glutathione delivery systems” Journal American College Nutrition 5(2):137-51, 1986). Reduced glutathione and its unique interaction with glutathione peroxidase moderate the effect of the —.OH radical, particularly in mitochondria, provided that the reduced glutathione can actually be delivered intracellularly. As the hydroxyl radical is a product of ionizing radiation and water, the ability to neutralize the hydroxyl radical, makes liposomal reduced glutathione an ideal candidate for use in the treatment of radiation exposure. Scavengers of the .OH radical have been demonstrated to protect mammalian animal cells against the damaging effects of radiation (Ewing D et al, “Radiation protection of in vitro mammalian cells: effects of hydroxyl radical scavengers on the slopes and shoulders of survival curves,” Radiation Res , Vol. 126(2):187-97, May 1991). The mechanism is related to the scavenging of the .OH radical as well as other intracellular mechanisms. An evaluation of 35 people with “post radiation syndrome” after exposure to substantial amounts of ionizing radiation (0.01-0.5 Gy) while participating in recovery work in Chernobyl demonstrated that they had a significant decrease in antioxidant defense with a decrease in the activity of glutathione peroxidase and deficiency of selenium.(22). While the concept that antioxidant support is needed there have been two major problems. The enzyme glutathione peroxidase, needed to manage the hydroxyl radical requires glutathione as a specific substrate (23). Until the availability of the present invention there has been no way to provide glutathione in a convenient form. Other antioxidants such as vitamin C and E may provide limited help in maintaining glutathione, but the chronic oxidative environment created by radiation exposure ultimately results in the loss of glutathione (24). The second problem is that since glutathione is not absorbed orally without the benefit of the liposome encapsulation of the present invention, there has not been a non-toxic oral alternative for providing prophylaxis or treatment that is effective and could be dispensed rapidly to a large population.

### OBJECTIVES OF THE INVENTION

The object of the invention is to offer a therapeutic agent and regimen for management of radiation exposures and ingestions that can be absorbed orally, and alternatively, transdermally, mucosally or by inhalation.

Another object of the invention is to offer a therapeutic agent that can be stored for extended periods of time without refrigeration and that can be absorbed orally, and alternatively, transdermally, mucosally or by inhalation.

Another object is to enable immediate treatment without fear of toxicity of individuals exposed to radiation without fear of compromising later desired treatments, particularly in instances of delayed diagnosis.

Another object is to present a non-toxic oral alternative (usually the only effective alternative is intravenous treatment) for providing that is effective and could be dispensed rapidly to a large population without prescription and without toxic effect from reasonable overdose.

### DESCRIPTION OF FIGURES

FIGS. 1A, 1B, and 1C show the results of (A) Urinary/(B) Fecal elimination and (C) tissue retention (expressed as percentage of administered dose) of IV administered Co-60 in Wistar-Han rats. The figures show the results in the control animals versus the results in the animals from the effect of single oral treatment with ReadiSorb™ liposomal reduced glutathione. FIG. 1A shows the number of the day post-exposure on the x-axis, and the y-axis shows the percentage of the administered dose excreted in the urine. FIG. 1B shows the number of the day post-exposure on the x-axis, and the y-axis shows the percentage of the administered dose excreted in the urine. FIG. 1C has the relative percentages on the y-axis of percentage of Co-60 administered dose remaining in tissue for liver, skeleton and blood respectively.

FIGS. 2A, 2B and 2C show (A) Urinary (B) Fecal elimination and (C) tissue retention respectively (expressed as percent of administered dose) of IV administered Co-60 in Fisher F344 rats: effect of repetitive treatment (once daily for 5 days) with ReadiSorb (oral) or non-formulated reduced glutathione (oral or IV). The x-axis in FIGS. 2A and 2B show the number of the day post-exposure. The y-axis in FIG. 2A shows the shows the percentage of administered dose of Co-60 excreted by urination. The y-axis in FIG. 2B shows the percentage of administered dose of Co-60 excreted fecally. The x-axis in FIG. 2C shows the relative percentages on the y-axis for each of the liver, kidney, skeleton, skin/hair and muscle. The y-axis in FIG. 2C shows the percentage of Co-60 remaining in tissue.

FIG. 3 shows the dramatic increase in actual glutathione levels in blood cells versus a placebo. The x-axis shows the elapsed time in minutes and the y-axis shows the increase of glutathione levels in red blood cells in % as between the placebo and liposomal reduced glutathione.

FIG. 4 shows the effect of liposomal reduced glutathione (ReadiSorb™) in enhancing the drainage of methyl mercury (MeHg) and Hg(II) from organs. If the blood mercury level rises, this is optimal because the mercury had to come from other than the blood supply, meaning the organs.

FIG. 5 shows the effect of a second liposomal reduced glutathione (ReadiSorb™) challenge. Here the effect appears to be that the blood stores are reduced and excreted.

FIG. 6 shows that the addition of liposomal reduced glutathione (Readisorb) to the regimen enhanced organ drainage and blood drainage of mercury.

FIG. 7 shows the positive effect of liposomal reduced glutathione (Readisorb) upon excretion of fecal methyl mercury.

### DESCRIPTION OF INVENTION

The invention may be packaged in the form liposomal reduced glutathione either by itself or in combination with additional decorporation agents for convenience of use. This would be particularly advantageous to individuals engaged in activities that require them to pack their supplies such as soldiers in the field or early responders to a suspected radionuclide tenor event. The example of soldiers typifies the situation in which availability of the invention for prophylaxis, early treatment or aggressive treatment of exposure to nuclear, biological or chemical weapons, including weapons of tenor, is important. The invention is also applicable to chelation and absorption of non-radioactive transition metals, particularly mercury and especially methyl mercury.

### Dosing

The inventors' recommend administration of Selenium 100 mcg to 200 mcg in addition to liposomal reduced glutathione to maintain and restore Selenium levels. Selenium may be administered as organic forms of selenium, such as selenomethionine and selenocysteine, as well as the inorganic forms of the mineral, like sodium selenite and selenate. There is no need to over-administer selenium, but in its absence, the function of glutathione peroxidase and the liposomal reduced glutathione will be attenuated.

Liposomal reduced glutathione: The preferred dosing schedule of the invention for the treatment of symptoms related to radiation exposures is 600 mg (1.5 teaspoons) of the invention to be taken at the first onset of symptoms or after a known exposure. A dose of 400 mg (1 teaspoon) to 600 mg is to be repeated each hour until symptoms are relieved. Once symptom relief is achieved, the dose is repeated immediately upon the return of symptoms. The anticipated amount to be taken is 1 to 2 ounces in 24 hours.

If symptoms recur in the following 24 hours the regimen may be repeated as stated.

1 ounce is 5.56 teaspoons.

1 teaspoon of the invention of oral liposomal encapsulated reduced glutathione contains approximately 440 mg liposomal reduced glutathione.

A preferred mode sets a suggested dose based on body weight. Most patients prefer liposomal reduced glutathione gently stirred into water, fruit juice or the non-alcoholic beverage of their choice.

Determine Individual Dose By Body Weight: For children

Under 30 lbs: ¼ teaspoon=100 mg liposomal reduced glutathione

30-60 lbs: ½ teaspoon=210 mg liposomal reduced glutathione

60-90 lbs: ¾ teaspoon=316 mg liposomal reduced glutathione

90-120 lbs: 1 teaspoon=422 mg liposomal reduced glutathione

120-150 lbs: 1½ teaspoon=630 mg liposomal reduced glutathione

Over 150 lbs: 1½ teaspoons=630 mg liposomal reduced glutathione

### Dosing Schedule for the Treatment Rapidly Developing Symptoms.

As stated, the initial dose should be according to body weight. For adults the dose is 1 and ½ teaspoon initially and repeat every 1 to 2 hours over 24 hour period.

The amount and frequency of doses may be decreased as the individual begins to improve. The period of treatment is usually 24 hours.

Doses for use for prophylaxis of exposure to radiation:
Adults liposomal reduced glutathione 600 mg three times a day, the invention should be used on a continuous basis until the severity of exposure and response to therapy can be assessed.
Children—should use a dose of liposomal reduced glutathione equivalent to 60 mg/Kg of body weight three times per day.

These doses should be continued for the duration of the exposure and for 2 to 4 weeks after radiation exposure. If the exposure is high dose radiation, the dosage should be continued for 6 months.

Dosage for use after radiation exposure: Adults—liposomal reduced glutathione 600 mg 4 times per day per day in two or four divided doses. Children—90 to 120 mg /Kg body weight daily in divided doses.

If symptoms of radiation toxicity are developing: Increase the dose to 1200 mg (teaspoons) to 4 times a day for a 70 kg man. If needed, the dosing may be increased to 2 ounces (5000 mg to 10,000 mg) per day.

### Further Detailed Description of the Preferred Embodiments

### Pharmacologic Compounds

Selenium will be the collective reference for selenium enhancing compounds in this description. Those compounds include Selenium which is normally referred to as Selenium, but in this invention, the term selenium, also includes the following: Selenium may administered as organic forms of selenium, such as selenomethionine and selenocysteine, as well as the inorganic forms of the mineral, like sodium selenite and selenate. As stated above, the inventors' recommend administration of Selenium 100 mcg to 200 mcg in addition to liposomal reduced glutathione to maintain and restore Selenium levels. Selenium may administered as organic forms of selenium, such as selenomethionine and selenocysteine, as well as the inorganic forms of the mineral, like sodium selenite and selenate. There is no need to over-administer selenium, but in its absence, the function of glutathione peroxidase and the liposomal reduced glutathione will be attenuated.

In the following examples the term EDTA is used to include the similar material, polyaminopolycarboxylic acid, as well as calcium disodium ethylenediaminetetraacetate (CaNa(2)EDTA), magnesium EDTA (MgEDTA) as well as calcium trisodium or zinc trisodium diethylenetriaminepentaacetate (CaNa(3)DTPA, ZnNa(3)DTPA) or any additional forms of the materials known as EDTA or DTPA.

Additional chelators that may be included in the preferred embodiment include Dimethylpropanesulfate (DMPS) and Dimercaptosuccinic acid (DMSA) and other vicinal dimercaptans or dithiols.

The preferred form of DMPS for decorporation is the liposomal encapsulation of DMPS manufactured using the methods described in the examples. The preferred method for usage is the combination of liposomal DMPS in combination with liposomal reduced glutathione. DMPS is water soluble and can be taken orally; however, it is known to be only 50% absorbed into the systemic circulation. The purpose of liposomal encapsulation is to increase the absorption of the DMPS in a fashion similar to the increased absorption observed for glutathione in case example 1.

### Dosing Schedule for Liposomal Encapsulated DMPS:

The single dose of DMPS contains 200 mg per 5.5 cc or per teaspoon of the material. The material is a liquid designed to be taken orally.

For acute radiation exposure: DMPS 100-200 mg oral 12 times a day. 1 teaspoon per os (orally) every 8 hours for 48 hours.

Less severe, or poisoning 100 mg 3-4 times daily for 48 hours initially, then the dose of 100 mg by be repeated every 12 hours for 3 days. Continued dosing will be determined by the condition of the patient.

For children, the appropriate dose would be 20 mg/kg.

During the use of oral liposomal DMPS, replacement of zinc will be needed to be instituted once the acute episode has been mediated and in any case 30 mg of zinc should be administered orally twice a day (taken 1 hour separate from the liposomal DMPS).

For treatment of orally ingested materials such as polonium-210 which can occur in poisonings via liquid ingestion, a combination of the plain, unformulated DMPS 100 mg and liposomal encapsulated DMPS 100 mg (½ teaspoon) may be taken simultaneously or in altered dosing. The object of using the combination is to take advantage of the fact that approximately 50% of the DMPS is not absorbed and to allow the binding of the radionuclide in the bowel. At the same time, the increased absorption allows the binding and removal of the radionuclide in the systemic system. The systemic absorption of DMPS is useful for radionuclides such as polonium-210 as this material is concentrated in soft tissues such as the reticuloendothelial system, spleen and liver. The preferred maximum dose for adults would be 250 mg.

The preferred dose for Prussian blue is 20 g (29) per day as an ameliorative along with the preferred dose of reduced liposomal glutathione.

For DTPA, the preferred dose would be either Ca-DTPA or Zn-DTPA 1 gram per day for 5 days. The Ca-DTPA form is preferred initially due to improved acceptance. For prolonged therapy, the Zn-DTPA form is preferred to avoid Zn deficiency, but is associated with a metallic taste. The preferred form for this embodiment is Ca-DTPA 1 gm per day for 5 days. Oral Zn 30 mg per day replacement is recommended.

The general preferred embodiment for EDTA, which has more particulars later in the discussion is EDTA Ca-EDTA 1.5 gms per day as it is tolerated well even after rapid administration. Oral Zn replacement, 30 mg per day is recommended. Up to 3.0 grams per day can be utilized. It has recently been observed that the rapid infusion of Ca-EDTA results in an increased excretion of metals such as mercury and may result in an increased excretion of radioactive materials at levels not previously associated with EDTA infusion.

For D-penicillamine (D-3-mercaptovaline), more generally referred to as Penicillamine, the appropriate regimen (in association with the liposomal reduced glutathione) is 500-750 mg daily in preferably two divided doses (Maximum daily dose=1.5 g).

The preferable timing of the divided dose for the convenience of the patient and to assist the patient in remembering to take the dose is that tablets should be taken upon waking and approximately 12 hours later before sleeping. Iron preparations, zinc or indigestion remedies should not be taken within 2 hours of penicillamine (in order to prevent reduction in absorption of penicillamine).

The appropriate dosage, in association with the liposomal reduced glutathione of IGF-1 is 60 mcg up to 120 mcg per day in preferably two divided doses, with 120 mcg per day the maximum dose.

For DHEA, the appropriate dosage (in association with the liposomal reduced glutathione) is 50 mg daily for duration of treatment protocol or maximum of 10 days

### Example 1

Liposomal Reduced Glutathione Drink or Spray 2500 mg Per Ounce

TABLE 1

| Substance | % w/w |
|---|---|
| Deionized Water | 74.4 |
| Glycerin | 15.00 |
| Lecithin | 1.50 |
| Citrus Seed Extract | 0.50 |
| Potassium Sorbate | 0.10 |
| Reduced Glutathione | 8.50 |

Or

| | |
|---|---|
| Deionized Water | 74.9 |
| Glycerin | 15.00 |
| Lecithin | 1.50 |
| Potassium Sorbate | 0.10 |
| Reduced Glutathione | 8.50 |

Or

| | |
|---|---|
| Deionized Water | 74.5 |
| Glycerin | 15.00 |
| Lecithin | 1.50 |
| Citrus Seed Extract | 0.50 |
| Reduced Glutathione | 8.50 |

Components lecithin (preferably hydrolyzed) and glycerin were commingled in a large volume flask and set aside for compounding. (Alternatively, in all of the embodiments where the glutathione (reduced) percentage is 8.5, the glutathione (reduced) percentage can be lowered to 8.25 with 0.25% tocopherol acetate added).

In a separate beaker, water, potassium sorbate and glutathione were mixed and heated to 50 degrees C., and the temperature maintained at 50° C. or slightly below.

The water mixture was added to the lipid mixture while vigorously mixing with a high speed, high shear homogenizing mixer at 750-1500 rpm for 30 minutes, and the temperature maintained at 50° C. or slightly below.

The homogenizer was stopped and the solution was placed on a magnetic plate, covered with parafilm and mixed with a magnetic stir bar until cooled to room temperature. As an alternative, potassium sorbate or citrus seed extract or other suitable antibiotic and antifungal agent is added and the solution was placed in appropriate dispenser for ingestion as a liquid or spray dispenser.

Analysis of the preparation under an optical light microscope with polarized light at 400.times. magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. Nos. 5,891,465, and 6,610,322, 6,726,924, and Keller, Ser. No. 11/588,086 published as US20070042032 are incorporated into this description.

A variation of the preferred embodiment of the invention is the addition of EDTA (ethylene diamine tetraacetic acid) 100 mg per ounce to be encapsulated in the liposome along with the glutathione.

### Example 1A

Liposomal reduced glutathione Drink or Spray 2500 mg Per Ounce or Form Suitable for Encapsulation or Gel

TABLE 1A

| Substance | % w/w |
|---|---|
| Deionized Water | 74.9 |
| Glycerin | 15.00 |
| Lecithin | 1.50 |
| Potassium Sorbate | 0.10 |
| Reduced Glutathione | 8.50 |

A lipid mixture having components lecithin (preferably hydrolyzed), and glycerin were commingled in a large volume flask and set aside for compounding.

In a separate beaker, a water mixture having water, potassium sorbate, glycerin, glutathione were mixed and heated to 50.degree. C, and the temperature maintained at 50° C. or slightly below.

The water mixture was added to the lipid mixture while vigorously mixing with a high speed, high shear homogenizing mixer at 750-1500 rpm for 30 minutes, and the temperature maintained at 50° C. or slightly below.

The homogenizer was stopped and the solution was placed on a magnetic stirring plate, covered with parafilm and mixed with a magnetic stir bar until cooled to room temperature. Normally, citrus seed extract would be added. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400 times magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. Nos. 5,891,465, and 6,610,322, 6,726,924, and Keller, Ser. No. 11/588,086 published as US20070042032 are incorporated into this description. The result of the preferred embodiment is a novel hydrolyzed liposomal reduced glutathione that does not need to be cold-stored to maintain stability, and can be administered orally, mucosally, dermally or intravenously in contrast to any other product in the marketplace, and is effective against radiation exposures.

For any of these examples sated and following, Phospholipon 90 (DPPC) could be substituted in the ingredient list for lecithin in certain circumstances.

### Example 2

Liposomal Reduced Glutathione Drink 1000 mg Per Ounce with EDTA in a Liposome 1000 mg Per Ounce

TABLE 2

| Substance | % w/w |
|---|---|
| Deionized Water | 76.3 |
| Glycerin | 15.00 |
| Lecithin | 1.50 |
| Citrus Seed Extract | 0.50 |
| Potassium Sorbate | 0.10 |
| Reduced Glutathione | 3.3 |
| EDTA | 3.30 |

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.

The liposome-capsule unit containing encapsulated material can be taken in addition to orally, used for topical unit-of-use application, or other routes of application such as intra-ocular, intranasal, rectal, or vaginal.

The composition of examples 1 and 2 may be utilized in the encapsulated embodiment of this invention.

Gelatin capsules have a lower tolerance to water on their interior and exterior. The usual water tolerance for a soft gel capsule is 10% on the interior. The concentration of water in a liposome formulation can range from 60-90% water. An essential component of the present invention is the formulation of a liposome with a relatively small amount of water, in the range of 5-10%. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% range.

The methods of manufacture described in Keller et al, U.S. Pat. Nos. 5,891,465, and 6,610,322, 6,726,924, and Keller, Ser. No. 11/588,086 published as US20070042032 are incorporated into this description.

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. Filling of gel caps on a large scale is best with the rotary die method or others such as the Norton capsule machine.

### Example 3

### Glutathione LipoCap Formulation

TABLE 3

| Substance | Ingredient concentration |
|---|---|
| Deionized Water | 4.0 |
| Glycerin | 15.00 |
| Lecithin | 2.0 |
| Potassium Sorbate | 0.20 |
| Reduced Glutathione | 89.8 |

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting one gram capsule contains 500 mg. Large scale manufacturing methods for filling gel caps, such as the rotary die process, are the preferred method for commercial applications.

Embodiment number three of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. Nos. 5,891,465, and 6,610,322, 6,726,924, and Keller, Ser. No. 11/588,086 published as US20070042032 are incorporated into this description.

The advantage of the Keller process and liposome is that most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of Pₐ (Pₐ references the surface packing parameter) between about 0.84 and 0.88, a Pᵥ (Pᵥ references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution. The methods of manufacture described in Keller et al, U.S. Pat. Nos. 5,891,465, and 6,610,322, 6,726,924, and Keller, Ser. No. 11/588,086 published as US20070042032 are incorporated into this description.

A variation of embodiment three is the combination of liposomal glutathione (reduced) and EDTA.

Additional variations of the method of manufacture of this embodiment of liposomal glutathione (reduced) and the compounds claimed in this invention, including EDTA or DTPA can be determined from the Keller art previously cited, especially Keller et al, U.S. Pat. No. 6,610,322.

### Case Example 1

A study of the effects of liposomal reduced glutathione was conducted at the Batelle Pacific Northwest National Laboratory and showing the surprising effects of the invention.

### ReadiSorb Pilot Decorporation Study

An initial 48-hr pilot study was conducted to evaluate the decorporation potential of ReadiSorb toward Co-60 in male Wistar-Han rats. Animals were restricted from food overnight prior to exposure. At the time of exposure, two groups of animals (n=6) received a single intravenous (IV) injection (0.2 mL) of Co-60 solution at 14.0±0.6 KBq dose in sterile saline via an indwelling jugular vein cannula. Immediately following IV injection, one group of animals received a single oral gavage dose of 0.5 mL ReadiSorb liposomal reduced glutathione (250 mg per kg BW). The second group of animals received the radionuclide without subsequent administration of the chelation material to serve as control group. Following dosing, animals were placed in Nalgene metabolism cages for separate collection of urine and feces. Animals were restricted from food for 1 hr after dosing with ReadiSorb. Animals were sacrificed 48 hr post radionuclide administration. At sacrifice, selected tissues (blood, liver and bone) were collected, weighed and counted for radioactivity using an automated Wallace 1480 (Perkin Elmer) gamma counter equipped with 3″ NaI(TI) crystal shielded detector. Gamma count data were normalized to percent administered dose after adjusting the grams of tissue collected for total organ mass.

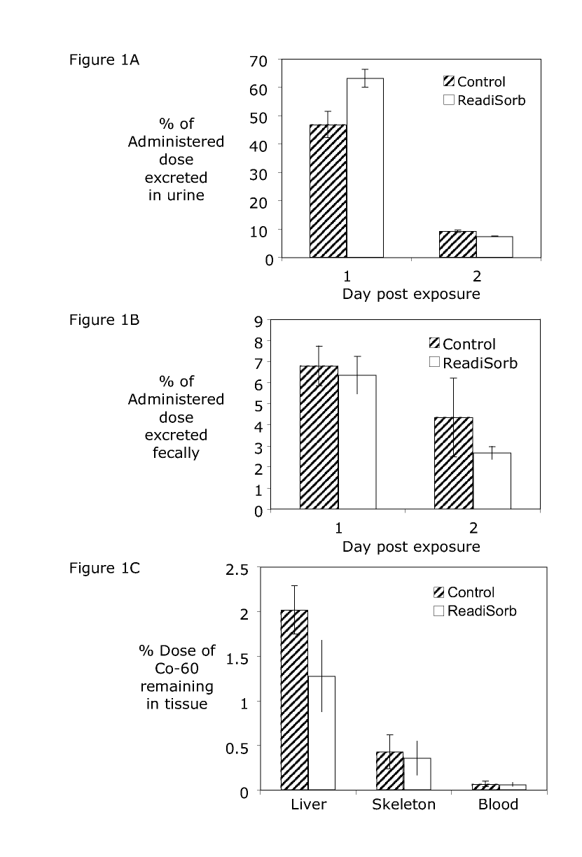

FIGS. 1A, 1B, and 1C show the results of (A) Urinary/(B) Fecal elimination and (C) tissue retention (expressed as percentage of administered dose) of IV administered Co-60 in Wistar-Han rats. The figures show the results in the control animals versus the results in the animals from the effect of single oral treatment with ReadiSorb(TM) liposomal reduced glutathione. FIG. 1A shows the number of the day post-exposure on the x-axis, and the y-axis shows the percentage of the administered dose excreted in the urine. FIG. 1B shows the number of the day post-exposure on the x-axis, and the y-axis shows the percentage of the administered dose excreted in the urine. FIG. 1C has the relative percentages on the y-axis of percentage of Co-60 administered dose remaining in tissue for liver, skeleton and blood respectively.

Co-60 elimination was monitored for 2 days following a single IV injection. The predominant route of excretion was via the urine, with about 47% and 9% of the administered radioactivity excreted in the urine within the first and second day post exposure, respectively (FIG. 1A). In comparison, fecal elimination accounted for approximately 6.8% and 4.4% of the administered radioactivity at the same time intervals (FIG. 1B). Administration of ReadiSorb liposomal reduced glutathione appeared to significantly accelerate urinal Co-60 elimination with 63.3% of the administered radioactivity excreted during the first day post exposure. Tissues collected post-mortem at day 2 post Co-60 exposure were found to contain measurable amounts of radioactivity with the highest levels in the liver (FIG. 1C). Consistent with the observed enhanced urinal elimination of Co-60, ReadiSorb reduced Co-60 level in liver by 37% (FIG. 1C); moderate reduction of radioactivity (17%) was observed in skeleton as well. The percent of administered radioactivity for the whole skeleton was calculated based on the femur data under the assumption that femur is representative of the bone as a whole (calculation assumes that total skeleton is approximately 7.3% of the body weight of the animal) (25).

Considering that only a single dose of ReadiSorb liposomal reduced glutathione was administered, results demonstrate significant benefit from the use of liposomal reduced glutathione for the decorporation of Co-60.

### Liposomal Reduced L-Glutathione Decorporation Studies.

Studies were conducted to test the decorporation efficacy of ReadiSorb toward Co-60 in male Fisher F344 rats over a 5-day period. The main goals of these studies were two-fold: (1) evaluate effect of repetitive oral administration of ReadiSorb on whole body retention of Co-60 and (2) compare the performance of orally administered liposomal reduced glutathione ReadiSorb with the performance of orally or IV administered non-formulated reduced glutathione. This study would also provide indirect data on systemic bioavailability of liposomal glutathione.

Animals were randomly assigned to one of 4 groups of n=4-12 animals per group. Animals received a single intravenous (IV) injection (0.2 mL) of Co-60 solution at 7.2±0.5 KBq dose in sterile saline via an indwelling jugular vein cannula. Immediately following IV injection, two groups of animals received the oral gavage dose of 0.5 mL ReadiSorb (300 mg per kg BW) liposomal reduced glutathione or plain, unprocessed reduced glutathione (300 mg per kg BW), the third group received the IV dose (0.3 mL) of reduced glutathione (95 mg per kg BW). Animals in the control group received only the single IV dose of Co-60 without consequent administration of chelation material. Following dosing, all animals were housed in Nalgene® metabolism cages and provided food and water. Repeat doses of chelation materials were administered to animals at 24-hour intervals until sacrifice. Urine and feces were collected per metabolism cage daily and analyzed for radioactivity. At sacrifice, selected tissues (liver, kidney, skin, muscle, and bone) were collected, weighed and counted for radioactivity. Gamma count data were normalized to percent administered dose after adjusting the grams of tissue collected for total organ mass.

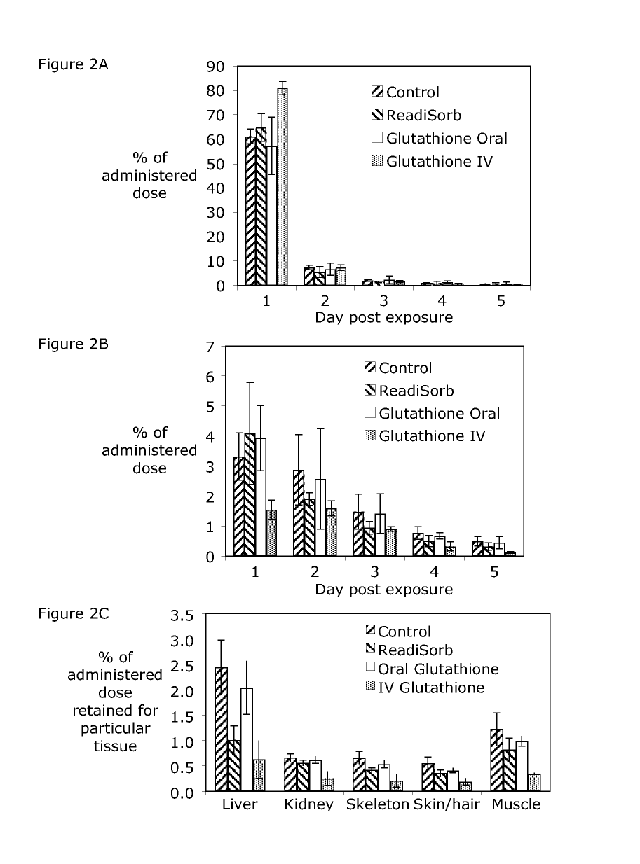

FIGS. 2A, 2B and 2C show (A) Urinary (B) Fecal elimination and (C) tissue retention respectively (expressed as percent of administered dose) of IV administered Co-60 in Fisher F344 rats: effect of repetitive treatment (once daily for 5 days) with ReadiSorb (oral) or non-formulated reduced glutathione (oral or IV). The x-axis in FIGS. 2A and 2B show the number of the day post-exposure. The y-axis in FIG. 2A shows the shows the percentage of administered dose of Co-60 excreted by urination. The y-axis in FIG. 2B shows the percentage of administered dose of Co-60 excreted fecally. The x-axis in FIG. 2C shows the relative percentages on the y-axis for each of the liver, kidney, skeleton, skin/hair and muscle. The y-axis in FIG. 2C shows the percentage of Co-60 remaining in tissue.

Examining the results, IV-administered Co-60 was eliminated predominantly through the kidney, with a cumulative 5-day urinary excretion of 72.3% of the administered dose (FIG. 2A, Control Group). This result is in excellent agreement with data by Gregus and Klaasen who reported that following single IV injection of 1 mg Co(II)/kg 72.6% of non-radioactive cobalt was excreted in urine by day 4.(26) Fecal excretion peaked at 3.3% at day one post exposure and gradually reduced to 0.5% at day 5. Liposomal reduced glutathione initially accelerated urinary and fecal excretion of Co-60 (FIGS. 2A,2B). As to cumulative urinary and fecal excretion of Co-60 over the 5-day period, liposomal reduced glutathione appeared to slightly enhance both urinary and fecal excretion of Co-60 (FIG. 2A,2B) versus control, but the difference was not statistically significant. In contrast, IV administration of the reduced glutathione markedly enhanced urinal elimination of Co-60 up to 81% of the administered dose (compared with 61% in the control group) at day one post exposure. This was accompanied by reduction of the fecal excretion, which was determined to be 1.5% of the administered dose versus 3.3% in the control group.

TABLE 4

| | Percent reduction of Co-60 relative to control in | | | | |
|---|---|---|---|---|---|
| Chelator | Liver | Kidney | Skeleton | Skin/Hair | Muscle |
| ReadiSorb | 59 | 15 | 36 | 36 | 33 |
| Oral | 17 | 7 | 19 | 26 | 20 |
| Glutathione | | | | | |
| IV | 74 | 62 | 69 | 66 | 70 |
| Glutathione | | | | | |

All tissues collected at sacrifice at day 5 post exposure were found to contain measurable amounts of radioactivity. In the control group, liver and muscle contained the largest amounts of Co-60 accounted for 2.4 and 1.2% of the administered dose, respectively. Kidney, skeleton, and skin each contained 0.55-0.65% of Co-60 dose. ReadiSorb (liposomal reduced glutathione) has demonstrated significant potential to prevent deposition of Co-60 in tissues (FIG. 2C) with the most significant 59% reduction of radioactivity observed in liver. Administration of ReadiSorb also resulted in 36% reduction of Co-60 in skeleton and skin, 33% reduction in muscle, and 15% in kidney (Table 4). Administration of reduced glutathione via IV route exhibited the most pronounced decorporation effect with the decrease of radioactivity ranging from 62% in kidney to 74% in liver. Oral administration of the non-formulated reduced glutathione was the least effective of the three methods.

These results suggest that reduced glutathione is a potent decorporation agent for Co-60 in the rat model. By analogy, reduced glutathione is a potent decorporation agent for RDD Radiotoxic Materials. The efficacy of the treatment is largely depends on the systemic availability of the glutathione. The obtained data support the prerequisite that oral delivery of liposomal glutathione markedly enhances its bioavailability compared to oral administration of non-formulated glutathione. However, the dosing regimen has to be adjusted to maximize concentration of glutathione in the blood and bioavailability studies are warranted prior to designing the treatment protocol.

In addition, these studies have revealed that administration of ReadiSorb liposomal reduced glutathione at high doses (up to 300 mg per kg BW (body weight)) and for prolonged periods of time (up to five days) induces no obvious signs of overt toxicity.

### Studies of Absorption and Function of Liposomal Glutathione:

The anti-oxidant and anti-atherogenic properties of liposomal glutathione were reviewed in a recent study.(23)The first part of the study demonstrated that liposomal glutathione has a significant anti-oxidant function in an in vitro human model of oxidative stress. In the second part of the study, liposomal glutathione demonstrated positive benefit in slowing the oxidative stress and chronic inflammatory components of atherosclerosis in an ApoE^{(-/-)} (knockout) mouse.(23) The ApoE^{(-/-)} mouse is a standard animal model of atherosclerosis.(27)

### Decorporation Studies:

As stated, these results suggest that liposomal reduced glutathione according to this invention is a potent decorporation agent for Co-60 in the rat model. By analogy, reduced glutathione is a potent decorporation agent for RDD Radiotoxic Materials and for mercury.

### Studies of Absorption and Function of Liposomal Glutathione:

The anti-oxidant and anti-atherogenic properties of liposomal glutathione were reviewed in a recent study.(23)The first part of the study demonstrated that liposomal glutathione has a significant anti-oxidant function in an in vitro human model of oxidative stress. In the second part of the study, liposomal reduced glutathione demonstrated positive benefit in slowing the oxidative stress and chronic inflammatory components of atherosclerosis in an ApoE^{(-/-)} (knockout) mouse.(23) The ApoE^{(-/-)} mouse is a standard animal model of atherosclerosis.(27)

The indirect data from the pilot study of Co-60 decorporation shows that liposomal glutathione is absorbed more efficiently than plain glutathione. The question of direct absorption of the liposomal formulation of glutathione is addressed in a pilot study of the pharmacokinetics of liposomal glutathione.

In Vitro Human Studies. A study reviewing the antioxidant and anti-atherogenic properties of liposomal glutathione done in the laboratory of Professor Michael Aviram has shown in-vitro benefits of liposomal glutathione using human blood.(23) The study was an in vitro experiment that evaluated the inhibition of oxidation of low density lipoprotein (LDL) using lipoproteins isolated from normolipidemic volunteers. The lipoproteins were exposed to CuSO₄ in a standard fashion. The addition of liposomal reduced glutathione inhibited the oxidation of LDL by 90% compared to the control.

Preliminary Pharmacokinetic Data from Two Subjects:

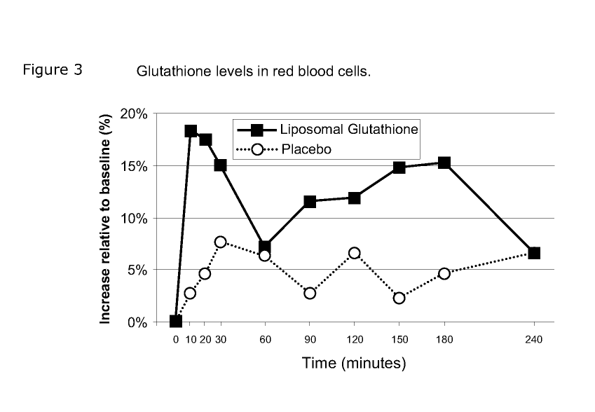

Preliminary data suggests that an oral bolus of liposomal glutathione (1.2 ounces or 3000 mg.) results in changes in red blood cell levels of glutathione in healthy individuals. The liposomal reduced glutathione levels in red blood cells in two healthy adults who were given a bolus of liposomal reduced glutathione one day (1.2 ounces, 3000 mg, liposomal reduced glutathione), and a placebo another day, with serial blood sampling for four hours, are presented in FIG. 3. The x-axis shows the elapsed time in minutes and the y-axis shows the increase of glutathione levels in red blood cells in % as between the placebo and liposomal reduced glutathione. The results show the dramatic increase in actual glutathione levels in blood cells versus a placebo.

### Availability-Generally Recognized as Safe:

The liposomal reduced glutathione preparation, carrying 420 mg/teaspoon, is sold as Readisorb™ by Your Energy Systems, LLC of Palo Alto, Calif., and is currently available as a dietary supplement. The material is made in a manufacturing site that follows cGMP (current Good Manufacturing Practice) protocol. The standard GMP testing of the material predicts a 2 year shelf life for this product, if stored at room temperature. The process used for the production of liposomal glutathione is scalable to large volumes.

The components of the liposomal reduced glutathione are classified as GRAS for food preparations. The liposomal reduced glutathione component has been proposed as GRAS, by its manufacturer, BioZone, Inc. from whom the liposomal formulation of the glutathione (reduced) is obtained and which manufacturer, in turn, utilizes the Keller art previously cited. Biozone is the preferable source for the liposomal reduced glutathione formulation proposed in this invention.

Glutathione in the plain powdered form has been available over the counter as a dietary supplement for over thirty years, however there are no studies referencing the use of oral glutathione except the study demonstrating lack of absorption of plain, powdered glutathione.(17) The liposomal reduced glutathione preparation has been used over the past three years by a number of physicians who are using it as part of protocols for the management of children with autism. No reactions have been reported.

### Summary Regarding Absorption and Function of Liposomal Glutathione.

The summation of the presented information confirms the clinical observation and the reported literature (Witschi) that plain, i.e. non-formulated glutathione is not absorbed well into mammalian systems. The mouse model shows that there is minimal function of plain glutathione when it is ingested at the same levels as the liposomal glutathione. In the mouse model, liposomal glutathione functioned to decorporate the amount of Co-60 residual in the liver at a rate that approached the function of intravenous glutathione. The same effect would be observed for RDD Radiotoxic Materials.

Management Information:

The invention also refers to not only combinations, but also methods of treatment, including serial or combined doses of the substances suggested above, and methods of manufacturing them in tandem. The concept of using growth hormone or IGF-1 for the management of radiation exposure has been investigated in an animal study.(28) (PMID 18049035) The combination of liposomal reduced glutathione and IGF-1 has been reviewed in Guilford, Serial No. published as US 20070053970. At present there is no way to administer IGF-1 in an oral form. The inclusion of IGF-1 in a liposomal encapsulation would facilitate its general use after radiation exposure.

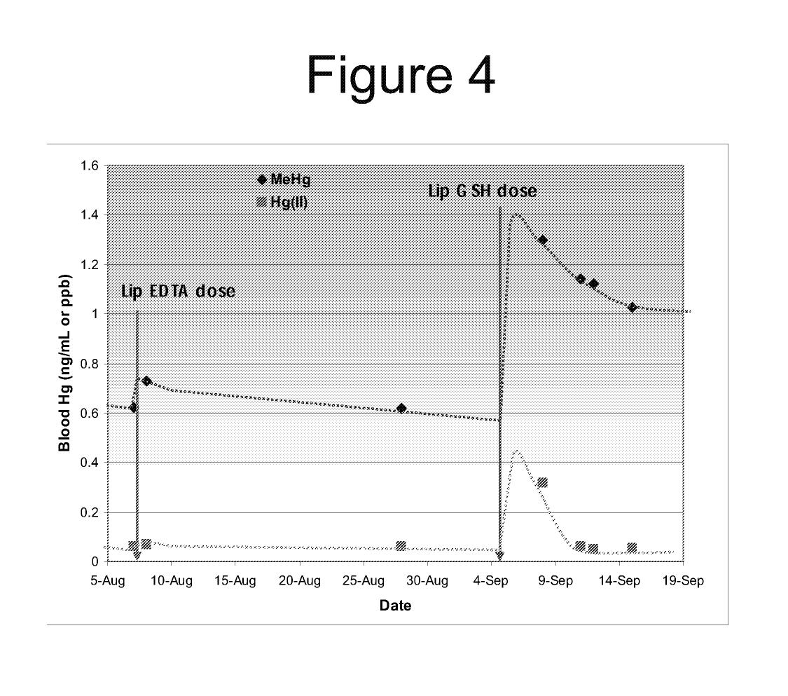

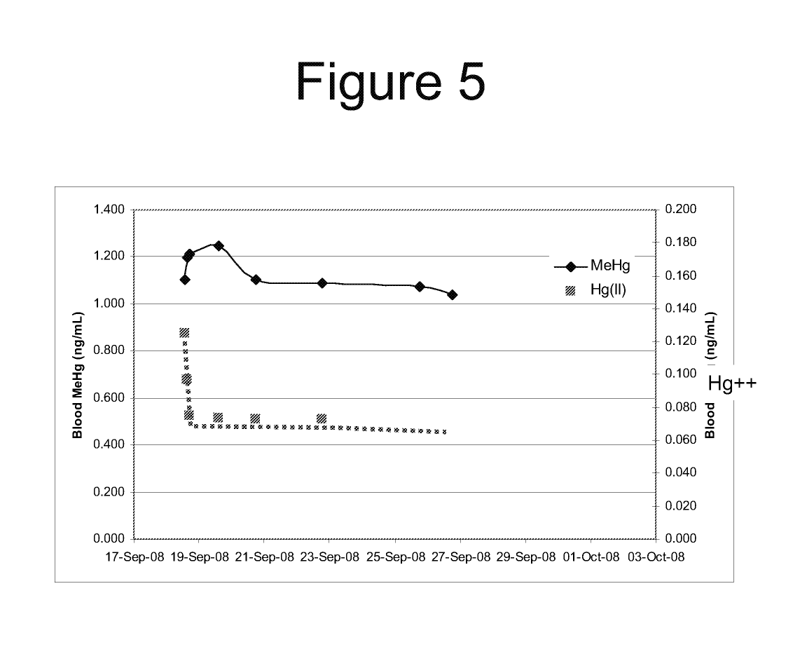

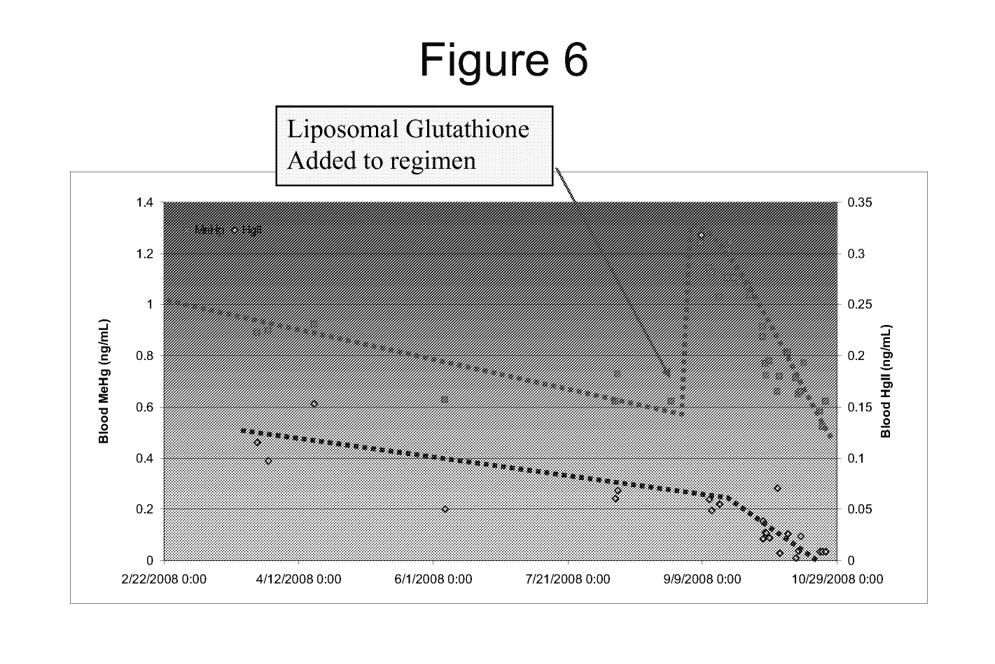

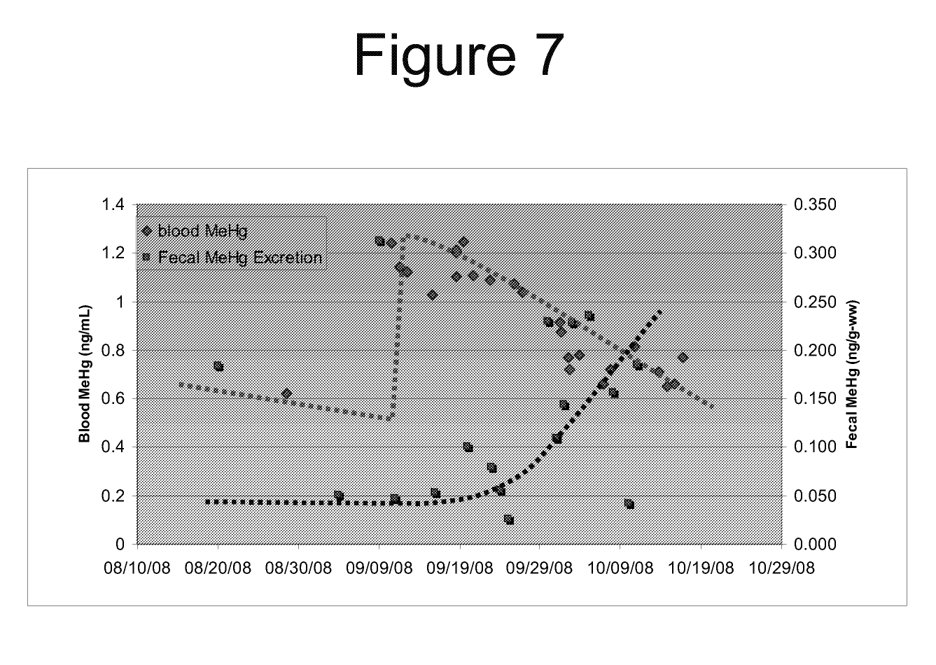

Referring to the results in FIGS. 4-7, liposomal reduced glutathione as described in this invention has positive metal extraction and chelation effects and enhances excretion out of the body, reinforcing the results of FIGS. 1-3 and showing that liposomal reduced glutathione as set forth in this invention can be used to remove RDD Radiotoxic materials and non-radioactive toxic mercury.

In one aspect the invention is a method of decorporating RDD radiotoxic materials from a contaminated mammal employing liposome encapsulated reduced glutathione (GSH). The method comprises oral administration of a liposome suspension of reduced glutathione) where at least 30% of the reduced glutathione is entrapped in the aqueous spaces of the liposomes and therefore protected from degradation in the GI tract. More preferably at least 50% is entrapped, and most preferably at least 70% is entrapped. The liposomes are preferably between about 100 and 1000 nm in diameter to optimize absorption through the GI tract. To increase sublingual absorption, and therefore more rapid uptake to the bloodstream, the liposomes may be sized so that some fraction of them are between about 20 to100 nm in diameter. Such sizing can be achieved by known methods, such as extrusion or filtration. The liposomes preferably comprise phospholipid, such as lecithin. To increase rigidity of the liposomes, and therefore decrease decomposition in the GI tract, the liposomes may also comprise cholesterol or other sterols such as plant sterols where the weight ratio of the phospholipid to sterol is between about 5:1 and 20:1, and more preferably about 10:1. The RDD radiotoxic materials may include Cobalt-60. The liposome suspension preferably also comprises selenium. The liposome suspension preferably also comprises other heavy metal chelators such as EDTA. Preferably at least 30% of the selenium and/or heavy metal chelators is entrapped in the aqueous spaces of the liposomes. More preferably at least 50% is entrapped, and most preferably at least 70% is entrapped. Preferably, the concentration of reduced glutathione in the entrapped aqueous space of the liposomes should be at least about 3.3 w/w per cent.

Preferably, the liposome suspension comprises a humectant. The humectant is preferably glycerin or sorbitol. The humectants preferably comprises between 2 and 25% of the w/w% of the liposome suspension. More preferably, the humectants comprises about 15%.

In another aspect, polysorbate 20 can be utilized as an emulsifier in conjunction with the liposome suspension.

In another aspect, the invention is a method of manufacturing a liposome suspension of reduced glutathione for use in decorporating RDD radiotoxic materials from a contaminated mammal. In this aspect, the resulting liposome suspension is as described above.

Dosing regimens described in this disclosure are based on liposome formulations where at least 30% of the reduced glutathione is entrapped in the aqueous spaces of the liposomes and therefore protected from degradation in the GI tract.

It should be noted that the examples herein were described using liposomal reduced glutathione that lack some of the preferred features of the invention. When the preferred features are incorporated into the composition, similar or improved results are expected.

The intent is to administer therapeutic doses of the substances referred to herein. The term “therapeutic dose” is intended to mean that amount of a drug or pharmaceutical agent that will elicit the biological or medical response of a tissue, a system, animal or human that is being sought by a researcher, veterinarian, medical doctor or other clinician. The term “prophylactically effective amount ” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. A consideration of these factors is well within the purview of the ordinarily skilled clinician for the purpose of determining the therapeutically effective or prophylactically effective amount. “Therapeutic window” is the therapeutic dose between the minimum amount of and the maximum amount of a pharmaceutical drug that will prevent or reduce the risk of occurrence of the biological or medical event that is sought to be prevented in a tissue, a system, animal or human by a researcher, veterinarian, medical doctor or other clinician. A “prophylactic dose” dose contemplates a dose that may be slightly less than the normal minimum amount of a pharmaceutical drug that will treat a biological or medical event that is sought to be overcome in a tissue, a system, animal or human by a researcher, veterinarian, medical doctor or other clinician, and addresses the problem of minimizing the toxicity of many drugs while having at least some of the drug present in the mammalian system to reduce the time before effective treatment by a drug begins.

The invention is not meant to be limited to the disclosures, including best mode of invention herein, and contemplates all equivalents to the invention and similar embodiments to the invention for humans and mammals and veterinary science. Equivalents include all pharmacologically active racemic mixtures, diastereomers and enantiomers of the listed compounds and their pharmacologically acceptable salts in any pharmaceutically acceptable carrier.

### References

1. Emergency Preparedness & Response: Facts About DTPA. 2006 [updated 2006; cited 2008 Apr. 2]; Available from: http://www.bt.cdc.gov/radiation/dtpa.asp.

2. Ip C, Lisk D J. Bioavailability of selenium from selenium-enriched garlic. Nutr Cancer. 1993; 20(2):129-37. Cited in PubMed; 8233978.

3. Ip C, Lisk D J. The attributes of selenium-enriched garlic in cancer prevention. Adv Exp Med Biol. 1996; 401:179-87. Cited in PubMed; 8886136.

4. Radiological Terrorism Fact Sheet: Radiological Weapons—“Dirty” Bombs. 2002 [updated 2002; cited]; Available from: http://bioterrorism.slu.edu/dirty/dirty.pdf.

5. Valentin J. Protecting people against radiation exposure in the event of a radiological attack. A report of The International Commission on Radiological Protection. Annals of the ICRP. 2005; 35(1):1-110, iii-iv. Cited in PubMed; 16164984.

6. Wood R, Sharp C, Gourmelon P, Le Guen B, Stradling G N, Taylor D M, et al. Decorporation Treatment—Medical Overview. Radiat Prot Dosimetry. 2000;87(1):51-6.

7. Taylor D, Stradling N, Ménétrier F. Biokinetics of radionuclides and treatment of accidental intakes. Radiat Prot Dosimetry. 2003;105(1-4):637-40.

8. Calcium-DTPA and Zinc-DTPA. [cited 2008 Apr. 2]; Available from: http://www.fda.gov/cder/drug/infopage/dtpa/.

9. Research CfDEa,. Frequently Asked Questions on Potassium Iodide (KI). U.S. Food and Drug Administration; [cited 2008 Apr. 2]; Available from: http://www.fda.gov/Cder/drugprepare/KI_Q&A.htm.

10. Research CfDEa. Prussian Blue (ferric hexacyanoferrate (II)) for Treatment of Internal Contamination with Thallium or Radioactive Cesium. [cited]; Available from: http://www.fda.gov/cder/drug/infopage/prussian_blue/default.htm.

11. NCRP. Management of Persons Accidentally Contaminated with Radionuclides Washington, DC: National Council Radiation Protection and Management; 1980.

12. Krezel A, Bal W. Structure-function relationships in glutathione and its analogues. Org Biomol Chem. 2003; 1(22):3885-90. Cited in PubMed; 14664379.

13. Krezel A, Bal W. Coordination chemistry of glutathione. Acta Biochim Pol. 1999; 46(3):567-80. Cited in PubMed; 10698265.

14. Ballatori N. Transport of toxic metals by molecular mimicry. Environ Health Perspect. 2002; 110 Suppl 5:689-94. Cited in PubMed; 12426113.

15. Smith R M, Martell A E. NIST Critically Selected Stability Constants of Metal Complexes, Reference Database 46, Version 5.0, US Department of Commerce, National Institute of Standards and Technology, Gaithersburg. Md., USA. 1998.

16. Clarkson T W, Vyas J B, Ballatori N. Mechanisms of mercury disposition in the body. Am J Ind Med. 2007; 50(10):757-64. Cited in PubMed; 17477364.

17. Witschi A, Reddy S, Stofer B, Lauterburg B H. The systemic availability of oral glutathione. European journal of clinical pharmacology. 1992; 43(6):667-9. Cited in PubMed; 1362956.

18. Readisorb. Your Energy systems, LLC; [cited 2008 Apr. 2]; Available from: http://www.readisorb.com.

19. Augustine A D, Gondre-Lewis T, McBride W, Miller L, Pellmar T C, Rockwell S. Animal models for radiation injury, protection and therapy. Radiat Res. 2005; 164(1):100-9. Cited in PubMed; 15966769.

20. Registry AfTSaD. What is cobalt? Division of Toxicology ToxFAQsTM [serial on the Internet]. 2004; (April): Available from: http://www.atsdr.cdc.gov/tfacts33.pdf.

20A. Marcus, C, Siegel, J and Sparks, R, Medical Management of Internally Radiocontaminated Patients, EMS Manuals and Protocols, published in 2006 by Los Angeles County Department of Health Services Emergency Medical Services Agency, 10100 Pioneer Blvd., Suite 200, Santa Fe Springs, Calif. 90670 also available online as of Jan. 23, 2009 at http://ems.dhs.lacounty.gov/ManualsProtocols/MMRSManual.pdf.

21. Beach B. A Critique (November 2006) MEDICAL MANAGEMENT OF INTERNALLY RADIOCONTAMINATED PATIENTS. The Applicability and Non-Applicability of the IR Manual to Nuclear War [serial on the Internet]. 2006: Available from: http://www.webpal.org/a_reconstruction/immediate/medical/nuclear/ir%20critique.htm#Drug.

22. Kumerova A O, Lece A G, Skesters A P, Orlikov G A, Seleznev J V, Rainsford K D. Antioxidant defense and trace element imbalance in patients with postradiation syndrome: first report on phase I studies. Biological trace element research. 2000;77(1):1-12. Cited in PubMed; 11097466.

23. Rosenblat M, Volkova N, Coleman R, Aviram M. Anti-oxidant and anti-atherogenic properties of liposomal glutathione: studies in vitro, and in the atherosclerotic apolipoprotein E-deficient mice. Atherosclerosis. 2007; 195(2):e61-8. Cited in PubMed; 17588583.

24. Ishii Y, Partridge C A, Del Vecchio P J, Malik A B. Tumor necrosis factor-alpha-mediated decrease in glutathione increases the sensitivity of pulmonary vascular endothelial cells to H202. The Journal of clinical investigation. 1992; 89(3):794-802. Cited in PubMed; 1541673.

25. Brown R P, Delp M D, Lindstedt S L, Rhomberg L R, Beliles R P. Physiological parameter values for physiologically based pharmacokinetic models. Toxicol Ind Health. 1997; 13(4):407-84. Cited in PubMed; 9249929.

26. Gregus Z, Klaassen C D. Disposition of metals in rats: a comparative study of fecal, urinary, and biliary excretion and tissue distribution of eighteen metals. Toxicol Appl Pharmacol. 1986; 85(1):24-38. Cited in PubMed; 3726885.

27. Zhang S H, Reddick R L, Piedrahita J A, Maeda N. Spontaneous hypercholesterolemia and arterial lesions in mice lacking apolipoprotein E. Science (New York, NY. 1992; 258(5081):468-71.

28. Matsouka P, Mylonas P, Papandoniou E, Dimitropoulou I, Floratou K, Alexandridis T, et al. Abdominal Radiation Initiates Apoptotic Mechanism in Rat Femur Bone Marrow Cells in vivo that is Reversed by IGF-1 Administration. J Radiat Res (Tokyo). 2007. Cited in PubMed; 18049035.

29. Keyes et al, Medical Response to Terrorism: Preparedness and Clinical Practice (Lippincott Williams & Wilkins 2004).

What is claimed is:

1. A pharmaceutical substance for lessening the tissue damaging effects of exposure to a radioactive bioterror weapon, including a terror weapon containing RDD radiotoxic materials, comprising:
in a pharmaceutically acceptable carrier, at least one therapeutic dose of reduced glutathione in a liposomal formulation being administrable by any one of three routes of administration, meaning administrable orally, dermally, transmucosally, or by inhalation.

2. The pharmaceutical substance according to claim 1, further comprising:
a therapeutic dose of Selenium.

3. The pharmaceutical substance according to claim 1, further comprising:
a therapeutic dose of metal-binding agents selected from the group of chemical substances having, DMPS, DTPA, EDTA, and penicillamine.

4. The pharmaceutical substance according to claim 1, further comprising:
a therapeutic dose of Insulin-like Growth Factor 1.

5. The pharmaceutical substance according to claim 1, further comprising:
a therapeutic dose of a polyaminopolycarboxylic acid selected from the group of alkali metals in combination with any one of DTPA or EDTA, or calcium disodium ethylenediaminetetraacetate (CaNa(2)EDTA).

6. The pharmaceutical substance according to claim 1, further comprising:
a therapeutic dose of a vicinal dimercaptan or dithiol selected from the group of DMPS or DMSA.

7. A pharmaceutical substance for lessening the tissue damaging effects of exposure to an RDD radiotoxic material or a non-radioactive transition metal, including mercury, comprising:
in a pharmaceutically acceptable carrier, at least one therapeutic dose of reduced glutathione in a liposomal formulation being administrable by any one of three routes of administration meaning administrable orally, dermally, transmucosally, or by inhalation.

8. The pharmaceutical substance according to claim 7, further comprising:
a therapeutic dose of Selenium.

9. The pharmaceutical substance according to claim 7, further comprising:
a therapeutic dose of a vicinal dimercaptan or dithiol such as DMPS or DMSA.

10. A pharmaceutical substance for lessening the tissue damaging effects of exposure to a radioactive bioterror weapon, including a terror weapon containing RDD radiotoxic materials, comprising:
in a pharmaceutically acceptable carrier, at least one therapeutic dose of reduced glutathione in a liposomal formulation being administrable by any one of three routes of administration meaning administrable orally, dermally, transmucosally, or by inhalation; and
said reduced glutathione in liposomal formulation being formulated by a process whose temperature does not exceed 50 degrees C., by preparing the formulation by utilizing the mixing of a first container of lecithin and glycerin, and a second container of components of at least deionized water and glutathione (reduced) at a temperature not in excess of 50 degrees, thereby resulting in reduced glutathione being capable of storage at room temperature for at least one month with at least 50% of original reduced glutathione.

11. The pharmaceutical substance according to claim 10, further comprising:
a therapeutic dose of Selenium.

12. The pharmaceutical substance according to claim 10, further comprising:
a therapeutic dose of metal-binding agents selected from the group of chemical substances having DMPS, DTPA, and EDTA.

13. The pharmaceutical substance according to claim 10, further comprising:
a therapeutic dose of Insulin-like Growth Factor 1.

14. The pharmaceutical substance according to claim 10, further comprising:
a therapeutic dose of a polyaminopolycarboxylic acid selected from the group of alkali metals in combination with any one of DTPA or EDTA, or calcium disodium ethylenediaminetetraacetate (CaNa(2)EDTA).

15. The pharmaceutical substance according to claim 10, further comprising:
a therapeutic dose of a vicinal dimercaptan or dithiol selected from the group of DMPS or DMSA.

16. A pharmaceutical substance for lessening the tissue damaging effects of exposure to an RDD radiotoxic material or a non-radioactive transition metal, including mercury, comprising:
in a pharmaceutically acceptable carrier, at least one therapeutic dose of reduced glutathione in a liposomal formulation being administrable by any one of three routes of administration meaning administrable orally, dermally, transmucosally, or by inhalation; and
said reduced glutathione in liposomal formulation being formulated by a process whose temperature does not exceed 50 degrees C., by preparing the formulation by utilizing the mixing of a first container of lecithin and glycerin, and a second container of components of at least deionized water and glutathione (reduced) at a temperature not in excess of 50 degrees, thereby resulting in reduced glutathione being capable of storage at room temperature for at least one month with at least 50% of original reduced glutathione.

17. The pharmaceutical substance according to claim 16, further comprising:
a therapeutic dose of Selenium.

18. The pharmaceutical substance according to claim 16, further comprising:
a therapeutic dose of a vicinal dimercaptan or dithiol such as DMPS or DMSA.

19. The pharmaceutical substance according to any one of claims 1 through 18, further comprising:
tocopherol acetate substituted for up to 0.25% by water/weight of said reduced glutathione in a liposomal formulation.
